(19) United States
(12) Patent Application Publication
(10) Pub. No.: US 2022/0370775 A1
(43) Pub. Date: Nov. 24, 2022

(54) HEEL CARE DEVICE

(71) Applicant: HealaHeel IP, George Town (KY)

(72) Inventor: Windsor William Woods, Toronto (CA)

(73) Assignee: HealaHeel IP, George Town (KY)

(21) Appl. No.: 17/772,231

(22) PCT Filed: Oct. 30, 2020
(86) PCT No.: PCT/CA2020/051467

§ 371 Date: Apr. 27, 2022

Related U.S. Application Data
(60) Provisional application No. 62/928,399, filed on Oct. 31, 2019.

(51) Int. Cl. A61M 35/00; A61F 13/06

(52) U.S. Cl. CPC A61M 35/10; A61F 13/067; A61M 2210/086

(57) ABSTRACT

A heel care device, having a cup section shaped to fit over and cover a portion of a subjects foot and a strap section configured to retain the cup section in place on the subjects foot. The strap section is made of a flexible, elastic material and the cup section is more rigid than the strap section.

### FIELD OF THE INVENTION

The present invention relates to foot care devices, in particular, to devices for applying moisturizer or medication to painful, infected, or dry and cracked skin on the foot.

### BACKGROUND

Many people suffer from dry and cracked skin or fungal infections of their feet, which, in severe cases, can be extremely painful and affect their mobility. Treatment for infections or dry and cracked skin on the foot typically includes the application of moisturizing or medicated creams, lotions, or ointments. Exfoliation and soaking of the affected area are also commonly used in connection with the application of moisturizing or medicated creams, lotions, and ointments.

It can be challenging to keep the moisturizing or medicated creams, lotions, or ointments in place on the affected area of the foot for long enough to provide effective treatment and/or moisturization. For example, when a person applies a medicated or moisturizing cream to their foot before going to sleep, they may move throughout the night and inadvertently remove the cream as their foot rubs against their bedsheets.

Many types of medicated cream, lotion, or ointment require prolonged contact with an affected area of a subject's skin. For example, athlete's foot is typically treated with medicated cream, lotion, or ointment, which is applied to the effected area of the foot.

Accordingly, there is a need for a device that will cover an affected area of a subject's foot to protect it and retain a moisturizing or medicated cream, lotion, or ointment in place to maximize its efficacy in treating the affected area of the subject's foot.

### SUMMARY OF THE INVENTION

A heel care device, according to the present invention, has a cup section shaped to fit over and cover a portion of a subject's foot and a strap section configured to retain the cup section in place on the subject's foot. The strap section is made of a flexible, elastic material and the cup section is more rigid than the strap section.

In another embodiment, the cup section and the strap section are integrally formed.

In another embodiment, the strap section comprises a continuous length of elastic material.

In another embodiment, the strap section comprises a buckle having a strap portion with a plurality of apertures and a buckle portion with a pin sized to engage one of the plurality of apertures.

In another embodiment, the portion of a subject's foot is the heel of the subject.

In another embodiment, the cup section has a groove adjacent to the perimeter edge of the cup section.

In another embodiment, the cup section has about the same flexibility in response to bending forces as the strap section and has more resistance to transverse tension and compression forces than the strap section.

In another embodiment, the cup section extends from the heel of the subject to the ball of the foot of the subject.

Various embodiments of the heel care device may be used with a moisturizing or medicated cream, lotion, or ointment to treat an affected area of a subject's foot.

### BRIEF DESCRIPTION OF THE DRAWINGS

In order that the invention may be more clearly understood, a preferred embodiment thereof will now be described in detail by way of example, with reference to the accompanying drawings, in which:

FIG. 1 is a perspective view of a heel care device, according to the present invention.

FIG. 2 is a top view of the heel care device.

FIG. 3 is a side view of the heel care device.

FIG. 4 is a front view of the heel care device.

FIG. 5 is a rear view of the heel care device.

FIG. 6 is a perspective view of another embodiment of the heel care device, illustrating a strap section with a buckle.

FIG. 7 is a top view of the heel care device of FIG. 6.

FIG. 8 is a right-side view of the heel care device of FIG. 6.

FIG. 9 is a left-side view of the heel care device of FIG. 6.

FIG. 10 is a front view of the heel care device of FIG. 6.

FIG. 11 is a rear view of the heel care device of FIG. 6.

FIG. 12 is a perspective view of another embodiment of the heel care device, illustrating a cup section extending from the heel to the ball of the subject's foot.

### DESCRIPTION OF THE PREFERRED EMBODIMENT

The heel care device, according to the present invention, is used with a moisturizing cream, lotion, or ointment to treat dry and cracked skin on a subject's foot. Alternatively, the device may be used with a medicated cream, lotion, or ointment to treat other conditions, such as athlete's foot. The device covers an area of a subject's foot and protects it while the medicated or moisturizing lotion treats the skin of the subject.

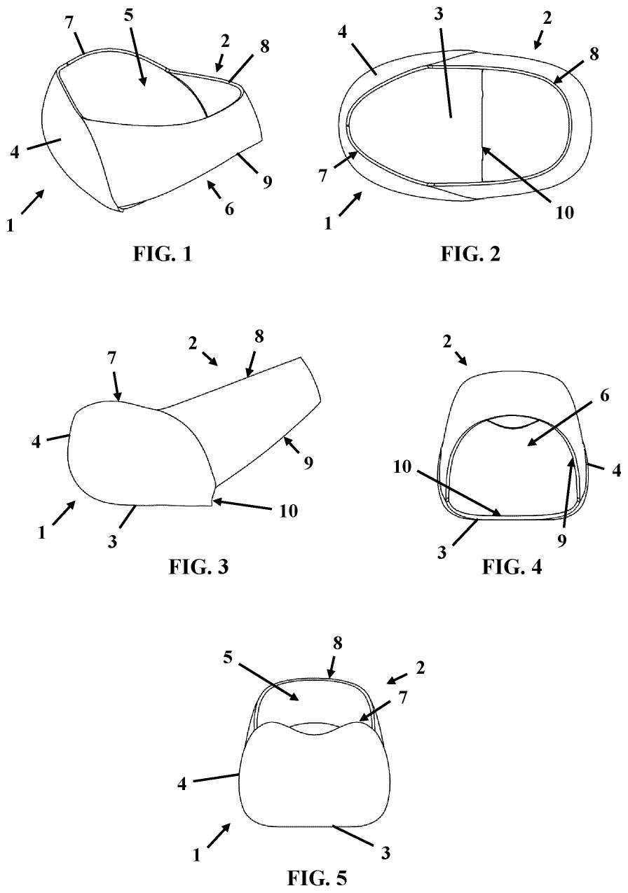

As shown in FIGS. 1-5, the heel care device has a cup section 1 and a strap section 2. The cup section 1 is shaped to fit over and cover an area of a subject's foot. The strap section 2 is shaped to extend about the subject's foot and hold it in place over the desired area of the subject's foot. Both the cup section 1 and the strap section 2 have a flexible sheet-like structure. The cup section 1 is flexible in response to a bending force, at an angle to the plane of the material, but resists deformation in response to transverse tension or compression forces along the plane of the material. As a result, the cup section 1 is able to accommodate variations in the dimensions and shape of the subject's foot within certain ranges. A set of standardized sizes of cup section 1 may thereby provide a comfortable fit to the majority of subjects' feet.

The strap section 2 is also flexible in response to a bending force, but unlike the cup section 1, is stretchable and readily deforms elastically in response to tension and compression forces along the plane of the material. This allows the subject to slide the device over the foot and into position over the desired area. The strap section 2 is sized so that it remains under tension while the subject is wearing the device and acts as an elastic band across the top of the subject's foot to retain the cup section 1 in place.

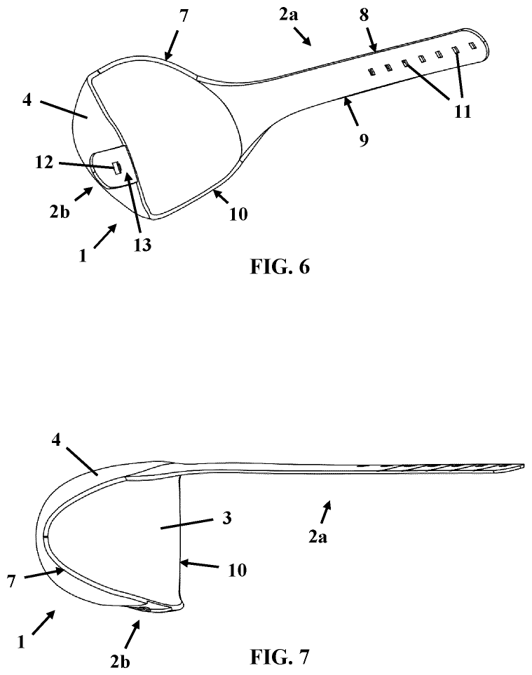

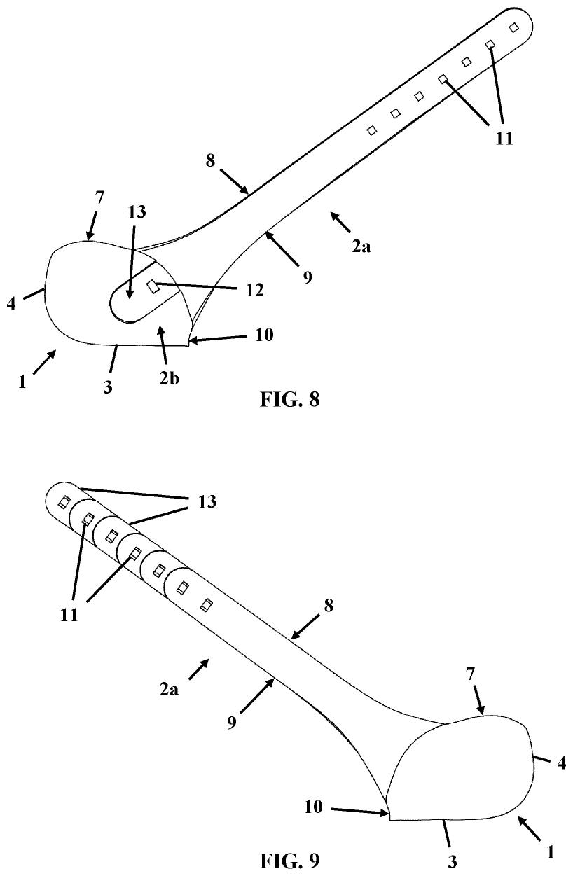

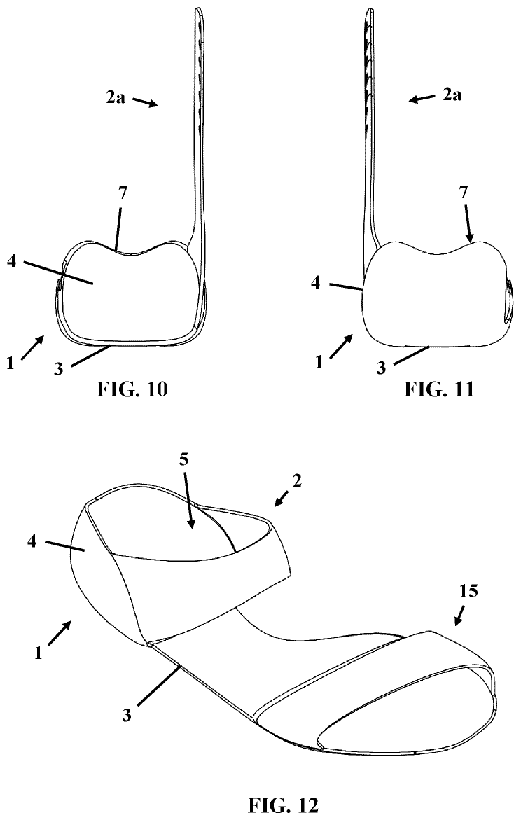

Alternatively, as shown in FIGS. 6-11, the strap section 2 may have a buckle that may be used to adjust the sizing of the strap section 2 for a more comfortable fit. Preferably, the buckle is a snap-type buckle with a strap portion 2a and a buckle portion 2b. As shown in FIGS. 6, 8, and 9, the strap portion 2a has a plurality of apertures 11 along a portion of its length to provide adjustability. The buckle portion 2b has a pin 12 shaped and sized to engage with one of the plurality of apertures 11 on the strap portion 2a to thereby secure the strap portion 2a to the buckle portion 2b. Preferably, the pin 12 is hook-shaped to securely retain the strap portion 2a in place. The buckle portion 2b may be recessed into the cup section 1 to provide a groove 13 shaped to receive the free end of the strap portion 2a. As shown in FIG. 9, the strap portion 2a may have a plurality of segments 14 that are readily removable with each segment 14 containing one or more apertures 11. The length of the strap portion 2a may thereby be adjusted to eliminate any extra length beyond what is required to fit the subject's foot.

The cup section 1 may be made from a variety of flexible materials, including silicone, rubber, thermoplastic elastomers (TPE), and plastic. Alternatively, the cup section 1 may be made from a rigid material, such as harder grade silicone, rubber, thermoplastic elastomers (TPE), or plastic. Where the cup section 1 is formed of a rigid material, a groove may be formed inside the perimeter edge 7 of the curved sidewall 4 and the front edge 10 of the floor 3 to assist in securing the cup section 1 in place on the subject's foot.

The strap section 2 may be made from the same flexible materials as the cup section 1, but include different additives to provide the desired degree of flexibility and elasticity in response to tension and compression forces. Preferably, the cup section 1 and the strap section 2 are formed integrally as a single monolithic unit.

Preferably, the cup section 1 is shaped to fit over the heel area of a subject's foot and covers the bottom and a portion of the sides of the subject's heel. Accordingly, the cup section 1 has a floor 3 and a curved sidewall 4, shaped to cover the bottom, rear, and sides of the heel of the subject. The strap section 2 extends between opposing sides of the curved sidewall 4, thereby defining a top opening 5 and a front opening 6. As shown in FIG. 2, the top opening 5 is defined by the perimeter edge 7 of the curved sidewall 4 and the top edge 8 of the strap section 2. As shown in FIG. 4, the front opening 6 is defined by the bottom edge 9 of the strap section 2 and the front edge 10 of the floor 3.

Alternatively, the cup section 1 may be shaped to fit over other areas of a subject's foot, such as the ball of the foot. As shown in FIG. 12, the floor 3 of the cup section 1 extends forwardly from the bottom edge 9 from the heel to the ball of the foot, where it is secured in place by a second strap section 15 that is sized to fit over the sides and top of the ball of the subject's foot.

The present invention has been described and illustrated with reference to an exemplary embodiment, however, it will be understood by those skilled in the art that various changes may be made and equivalents may be substituted for elements thereof without departing from the scope of the invention as set out herein. Therefore, it is intended that the invention not be limited to the particular embodiments disclosed herein.

What is claimed is:

1. A heel care device, comprising a cup section shaped to fit over and cover a portion of a subject's foot and a strap section configured to retain the cup section in place on the subject's foot, wherein the strap section is made of a flexible, elastic material and the cup section is more rigid than the strap section.

2. The heel care device of claim 1, wherein the cup section and the strap section are integrally formed.

3. The heel care device of claim 2, wherein the strap section comprises a length of elastic material.

4. The heel care device of claim 2, wherein the strap section comprises a buckle having a strap portion with a plurality of apertures and a buckle portion with a pin sized to engage one of the plurality of apertures.

5. The heel care device of claim 4, wherein the strap portion comprises a plurality of removable segments.

6. The heel care device of claim 2, wherein the portion of the subject's foot is the heel of the subject's foot.

7. The heel care device of claim 6, wherein the cup section has a perimeter edge and a groove adjacent the perimeter edge of the cup section.

8. The heel care device of claim 2, wherein the cup section has about the same flexibility in response to bending forces as the strap section and has more resistance to transverse tension and compression forces than the strap section.

9. The heel care device of claim 2, wherein the cup section extends from the heel of the subject to the ball of the foot of the subject.

10. The heel care device of claim 9, comprising a second strap section positioned over the ball of the foot of the subject.
